(19) United States
(12) Patent Application Publication
(10) Pub. No.: US 2018/0221546 A1
(43) Pub. Date: Aug. 9, 2018

(54) IMPLANTABLE ORTHOPEDIC DEVICES HAVING ANTIMICROBIAL COATINGS

(71) Applicant: TELEFLEX MEDICAL INCORPORATED, MORRISVILLE, NC (US)

(72) Inventors: NISHA GUPTA, READING, PA (US); KAMNA GIARE-PATEL, READING, PA (US); CHUANTING YOU, READING, PA (US)

(21) Appl. No.: 15/746,990

(22) PCT Filed: Jul. 22, 2016
(86) PCT No.: PCT/US16/43533

§ 371 Date: Jan. 23, 2018

Related U.S. Application Data
(60) Provisional application No. 62/196,429, filed on Jul. 24, 2015.

(51) Int. Cl. A61L 31/16; A61L 31/10; A61L 31/08; A61L 31/06; A61L 27/54; A61L 27/34; A61L 27/28; A61L 27/18

(52) U.S. Cl. CPC A61L 31/16; A61L 2430/34; A61L 31/08; A61L 31/06; A61L 27/54; A61L 27/34; A61L 27/28; A61L 27/18; A61L 2300/404; A61L 2300/206; A61L 2300/606; A61L 2420/06; A61L 2300/602; A61L 2430/02; A61L 31/10

(57) ABSTRACT

An implantable orthopedic device having an antimicrobial coating on at least one surface thereof is disclosed. The antimicrobial coating includes alexidine and a carrier polymer.

### TECHNICAL FIELD

The present disclosure relates generally to orthopedic devices, and more particularly to implantable orthopedic devices treated with antimicrobial coatings containing alexidine to prevent infection.

### BACKGROUND

Implanted orthopedic devices are widespread among the population today. Orthopedic devices are used to replace missing joints or bone, for fixation of long bone fractures and deformities, for replacement of arthritic joints, and for other orthopedic and maxillofacial applications. Although these devices are heavily disinfected or sterilized prior to implantation, many orthopedic devices nonetheless cause serious infections in patients after they are implanted in the body. Infections of orthopedic fracture and reconstructive devices occur in approximately 5% of cases and total about 100,000 cases per year in the United States alone. Infectious agents such as Staphylococcus epidermidis and Staphylococcus aureus, gram-negative bacilli and Candida species (a group of fungal agents) are largely responsible for the infections associated with orthopedic devices.

Orthopedic implant-associated infections pose serious health risks and complications for patients. If the infection is not detected early and successfully treated, the infection will progress requiring removal of the orthopedic device. A rigorous and prolonged regimen of antibiotics is usually administered to the patient to rid them of the infection. A replacement orthopedic device may be safely re-implanted only after the infection has been eliminated. Thus orthopedic implant-associated infections are a substantial healthcare burden, and leads to prolonged patient suffering, and substantial morbidity and even mortality.

Different approaches have been used to prevent the infections associated with implanted orthopedic devices. For example, one approach involves coating the orthopedic device with an antimicrobial coating. The antimicrobial coating includes an antimicrobial agent and must be able to maintain a sufficient antimicrobial effect for the duration that the orthopedic device is implanted within the patient.

Chlorhexidine is commonly used as the antimicrobial agent in many antimicrobial coatings for implantable medical devices. Although chlorhexidine has been useful to some extent in medical devices, there are some serious drawbacks to chlorhexidine. For example, it is known that chlorhexidine has the ability to function as a sensitizing agent, and in rare cases it can trigger immediate hypersensitivity in the form of acute anaphylaxis. Another drawback is that chlorhexidine must be present in high concentrations in order to function as a wide spectrum antimicrobial. Such concentrations of chlorhexidine may cause skin irritation or allergic reactions in some patients. Additionally, chlorhexidine may not be as effective against some microorganisms and/or may not kill microorganisms quickly. Therefore, there is an unmet need for an improved antimicrobial composition having a higher level of antimicrobial activity and lower toxicity to the patient's tissue.

Alexidine is a disinfectant that is widely used as an antimicrobial in rinse solutions for oral and ophthalmic (for example, for contact lens cleaning and disinfecting) applications, and has been commercialized in various products, typically at levels of about 100 ppm or less for use with soft contact lenses. As an oral disinfectant, typical concentration of alexidine is about 1%. Generally, it is desirable to provide the lowest possible level of antimicrobial that is consistent with reliable disinfection in order to provide a generous margin for safety and comfort. To date, alexidine, has not been used as an antimicrobial agent in antimicrobial coatings for implantable medical devices and orthopedic devices.

Both alexidine and chlorhexidine are antimicrobial agents known as bis-biguanides. Both antimicrobial agents possess the biguanide and the hexamethylene structures. Alexidine however, differs from chlorhexidine by possessing ethyl-hexyl end groups instead of chlorophenyl end groups. Due to this structural difference, alexidine is shown to produce lipid phase separation and domains in the cytoplasmic membrane of microbes. The domain formation in the microbial membrane allows alexidine to cause significantly faster alteration in membrane permeability leading to more rapid bactericidal effect as compared to chlorhexidine. The rapid microbial action of alexidine makes it especially beneficial in a skin disinfectant composition which may get utilized in situations requiring quick disinfection (like skin preparation prior to an emergency trauma surgery). Alexidine has also shown to promote apoptosis as an anti-cancer agent and possess anti-inflammatory, and antidiabetic properties, which can aid in rapid wound healing. Furthermore, Alexidine is also shown to have significantly lower risk of causing IgE (Immunoglobulin E) mediated hypersensitivity as compared to chlorhexidine.

Conventional antimicrobial coatings for implantable orthopedic devices are often inadequate and may still lead to infection. Therefore, improved antimicrobial coatings and implantable orthopedic devices are needed.

Accordingly, the implantable orthopedic devices and antimicrobial coatings disclosed herein are directed at overcoming one or more of these disadvantages in currently available orthopedic devices.

### SUMMARY

In accordance with one aspect of the disclosure, an implantable orthopedic device having an antimicrobial coating on at least one surface thereof is disclosed. The antimicrobial coating includes alexidine and a carrier polymer.

### DETAILED DESCRIPTION

Before the present methods and devices are disclosed and described, it is to be understood that the methods and devices are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

As used herein, the term “alexidine” includes alexidine, alexidine base, alexidine hydrochloride, alexidine dihydrochloride, alexidine monoacetate, alexidine diacetate, alexidine gluconate, alexidine digluconate and mixtures thereof. In general, the alexidine used in the antimicrobial composition may be prepared by any of the processes known in the art for manufacturing alexidine.

As used herein, the term or phrase “antimicrobial agent” may, in one aspect, refer to, without limitation, agent(s) that are responsible for, or cause the destruction and removal of viable microorganisms from a material including the biofilms and spores of the microorganisms. The antimicrobial agent may, also without limitation, refer to agents that effect a reduction of viable microorganisms and their spores and does not necessarily imply the complete removal of all viable microorganisms and their spores.

As used herein, the term “hypoallergenic” refers to a reduced allergic reaction or a reduced tendency to trigger hypersensitivity responses to allergens and may be mediated by IgE (Immunoglobulin E) antibodies.

As used herein, the term “orthopedic device” refers to medical devices that are used in orthopedic applications and may include without limitation rods, screws, pins, anchors, cages, and combinations thereof.

As used herein, the term “implantable” refers to an orthopedic device to be positioned partially or wholly at a location within a body, such as within a body vessel. Additionally, the terms “implantation” and “implanted” refer to the positioning of a medical device at a location, partially or wholly, within a body, such as within a body vessel.

As used herein, the terms “minimum inhibitory concentration” and “MIC” are used interchangeably and refer to the minimum concentration of an antibacterial agent in a given culture medium below which bacterial growth is not inhibited.,

As used herein, the terms “minimum bactericidal concentration” or “MBC” are used interchangeably and refer to the minimum concentration of an antibacterial agent in a given culture medium below which bacterial growth is not eliminated.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and devices may be understood more readily by reference to the following detailed description of preferred embodiments and the Examples included therein and to the Figures and their previous and following description.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The present disclosure makes use of alexidine in an antimicrobial coating that is used to coat at least one surface of an implantable orthopedic device. In certain aspects of the disclosure, the antimicrobial coating comprises alexidine as an antimicrobial agent and a carrier polymer.

The duration of implantation of the orthopedic device disclosed herein may be permanent or may intend to remain in place for the remaining life span of the patient or until the orthopedic device is physically removed from the patient.

The implantable orthopedic devices and the antimicrobial coatings disclosed herein show surprising and unexpected broad spectrum activity against various microorganisms. In particular, the antimicrobial effects obtained from antimicrobial coatings of the present disclosure, which include alexidine far exceed the results obtained from comparative antimicrobial coatings, which include chlorhexidine.

In one aspect, the antimicrobial coating has a broad spectrum antimicrobial effect against the gram positive bacteria, gram negative bacteria, and fungal pathogens responsible for infections. For example, the antimicrobial coating is effective against gram positive bacteria such as Staphylococcus aureus, gram negative bacteria such as Pseudomonas aeruginosa or fungi such as Candida albicans in both planktonic and biofilm forms, and to various extents. Therefore, methods of using the antimicrobial coating and the implantable orthopedic device described herein may be provided for the prevention and treatment of infections caused by these microorganisms.

In certain aspects of the present disclosure, the antimicrobial coating of the present disclosure may provide immediate and sustained delivery of alexidine to the tissues surrounding the implantable orthopedic device. Therefore, use of these implantable orthopedic devices may be effective in protecting the patient's body against pathogenic organisms.

The antimicrobial coating may further include various therapeutic agents. In one aspect, the therapeutic agents may include, without limitation an antibiotic, anaesthetic, analgesic, anti-inflammatory agent, bone density increasing agents, or mixtures thereof. In one aspect, the antimicrobial coating may improve bone density. In another aspect, the antimicrobial coating may promote wound healing. Wound healing may be achieved through the use of alexidine alone or the incorporation of other suitable agents into the antimicrobial coating known in the art to promote wound healing.

A surprising and unexpected finding of the antimicrobial composition disclosed herein is that it has been shown to be hypoallergenic, in particular as compared to antimicrobial compositions based on chlorhexidine. In another aspect, the antimicrobial composition may also be less likely to cause adverse reactions such as hypersensitivity and allergy. Methods and devices for the detection of allergic reactions and responses are described in U.S. Patent Application Publication No. 2014/0187892, the contents of which are incorporated herein by reference in their entirety. In certain aspects, the antimicrobial composition may also aid in reducing inflammatory responses such as erythema, phlebitis, and intimal hyperplasia.

Alexidine

The antimicrobial coating may include one or more of alexidine, alexidine base, alexidine hydrochloride, alexidine drochloride, alexidine monoacetate, alexidine diacetate, alexidine gluconate, alexidine digluconate and mixtures thereof. In general, the alexidine used in the antimicrobial coating may be prepared by any of the processes known in the art for manufacturing alexidine.

One advantage of the antimicrobial coating of the present disclosure is that a greater antimicrobial effect is achieved using a lower concentration of alexidine than other antimicrobial agents, such as chlorhexidine. In one aspect, the antimicrobial coating may have a concentration ranging from 0.0001 wt % to 4.0 wt % of alexidine. In another aspect, the antimicrobial coating may have a concentration ranging from 0.01 wt % to 2.0 wt % of alexidine. In another aspect, the antimicrobial coating may have a concentration of at least about 0.05 wt % of alexidine. The concentration of alexidine in the antimicrobial coating, however, is not limited in the present disclosure. The preferred amount of the antimicrobial coating on the orthopedic device may vary, depending on the nature of the orthopedic device and the nature of the implantation area.

In certain aspects of the present disclosure, the antimicrobial coating may not include chlorhexidine, triclosan, or silver. For example, in some aspects alexidine may be the only antimicrobial agent present in the antimicrobial coating.

Solvent

In certain aspects of the disclosure, a solvent may be used in the antimicrobial coating. The solvent may include water, an organic solvent, or any combination thereof. Suitable organic solvents, for example, may include without limitation, alcohol, dimethyl formamide, tetrahydrofuran (THF), ethyl acetate, butyl acetate, acetone, methyl ethyl ketone (MEK), citric acid, or mixtures thereof. Preferably, the solvent is one in which both the carrier polymer and alexidine are soluble. In one aspect according to the disclosure, the solvent used in the antimicrobial coating is an alcohol, such as isopropanol, methanol or ethanol or mixtures thereof. More than one solvent may be used in the antimicrobial coating. For example, in certain aspects, the solvent may comprise tetrahydrofuran (THF) and methanol, THF and ethanol, or THF and isopropyl alcohol, or THF and citric acid, or THF and isopropyl alcohol and citric acid.

The Carrier Polymer

In one aspect of the disclosure, the antimicrobial coating includes a carrier polymer. The carrier polymer generally includes a polymer that is soluble in alexidine. The carrier polymer may also be a biocompatible polymer that does not have any detrimental effect on the antimicrobial properties of alexidine. Furthermore, the carrier polymer may be a polymer that does not adversely affect the integrity of the orthopedic device in any manner. Suitable carrier polymers include without limitation, polyurethane, polypropylene, polyester, cellulose, poly(methyl methacrylate), acrylate, or combinations, thereof. In one aspect of the present disclosure, the carrier polymer is polyurethane.

Orthopedic Device

Particular orthopedic devices especially suited for application of the antimicrobial coatings of this disclosure include, without limitation orthopedic implants such as joint prostheses, screws, nails, nuts, bolts, plates, rods, pins, wires, inserters, osteoports, halo systems and other orthopedic devices used for stabilization or fixation of spinal and long bone fractures or disarticulations.

In certain aspects of the present disclosure, the orthopedic device may be composed of a metallic material, a non-metallic material such as a polymer material or a ceramic, or a combination thereof. Suitable metallic materials may include for example, stainless steel, titanium, chromium, cobalt and alloys thereof. Suitable polymer materials or non-metallic materials may include rubber, plastic, nylon, silicone, polyurethane, polyethylene, polyvinyl chloride, polytetrafluoroethylene tetraphthalate, polyethylene tetraphthalate, polytetrafluoroethylene, latex, and elastomers.

Methods

The antimicrobial coatings of the present disclosure may be prepared by any means known to those skilled in the art. For example, an antimicrobial coating solution may be prepared by mixing the alexidine and the carrier polymer with a solvent.

In certain aspects, the antimicrobial coating solution may then be applied to at least portion of the orthopedic device, and then allowing the coating solution to dry or cure to form the antimicrobial coating. The coating solution may be applied to the orthopedic device using any means known to those skilled in the art. In one aspect of the present disclosure, the antimicrobial coating solution may be sprayed onto surfaces of the orthopedic device. In other aspects, the orthopedic device may be dipped into the antimicrobial coating solution to form a coating, or may be brush coated, die coated, wiped, painted, or rolled onto the surfaces of the orthopedic device. In yet other aspects, extrusion methods may be useful to form either an antimicrobial layer on the orthopedic device or for bulk distribution of alexidine in the device. Any of these techniques or methods of applying the antimicrobial coating solution may be used in combination and/or repeated multiple s to form the desired antimicrobial coating.

In another aspect, the orthopedic device may be soaked in the antimicrobial coating solution for a period of time of about 5 seconds to about 5 minutes. In another aspect, the orthopedic device may be soaked in the antimicrobial coating solution for a period of time of about 2 seconds to about 2 minutes. In certain aspects, the orthopedic device is soaked in the antimicrobial coating solution for at least 4 seconds. However, the orthopedic device may be soaked in the antimicrobial coating solution for longer periods of time without adversely affecting the integrity of the orthopedic device. One advantage of the present disclosure is that the antimicrobial coating composition is a rapid disinfectant. This advantage is particularly valuable during orthopedic implant procedures where it is necessary to immediately facilitate sterilization and/or disinfection of the orthopedic implant itself, the implantation site and also its surroundings.

In certain aspects of the present disclosure, the orthopedic device may be dried at room temperature such that the solvent evaporates. In one aspect, the orthopedic device may be dried by removing the solvent from the antimicrobial coating composition. In another aspect, the solvent may be removed from the antimicrobial coating composition and an amount of alexidine may remain on a surface of the orthopedic device. The remaining amount of alexidine on the orthopedic device may provide an antimicrobial effect to the orthopedic device, which will serve to further prevent infection during the orthopedic procedure and in some cases, after the orthopedic procedure.

The alexidine may remain on the surface of the orthopedic device in its free form. Alternatively, the alexidine may become embedded in the matrix of the carrier polymer, which may provide a longer term antimicrobial effect for the patient through the orthopedic device. In certain aspects of the disclosure, the antimicrobial coating composition may be infused, absorbed, penetrated, coated, adhered into or onto a surface of the orthopedic device.

Abbreviations

The abbreviations used in the examples are as follows:

| | |
|---|---|
| MBC | Minimum Bactericidal Concentration |
| MIC | Minimum Inhibitory Concentration |
| MBC | Minimum Bactericidal Concentration |
| THF | Tetrahydrofuran |
| TNTC | Number of microbial colonies were Too Numerous To Count |

### EXAMPLES

Although the examples of the present invention will be set forth below, it will become apparent to anyone skilled in the art that the present invention is not limited by them and that various alterations and modifications may be made within the scope of the appended claims.

### Example 1

### Composition of Antimicrobial Solution Containing Chlorhexidine

An antimicrobial solution was prepared having the formulation shown in Table A.

TABLE A

| Ingredients | Amount (%) |
|---|---|
| Chlorhexidine | 2 |
| Water | 88 |
| Ethylene glycol | 10 |

### Example 2

### Composition of Antimicrobial Solution Containing Alexidine

An antimicrobial solution was prepared having the formulation shown in Table B.

TABLE B

| Ingredients | Amount (%) |
|---|---|
| Alexidine | 0.5 |
| Water | 89.5 |
| Ethylene glycol | 10 |

### Example 3

### Composition to Make Antimicrobial Coating for an Orthopedic Device

A coating solution having the formulation shown in Table C was prepared for application on orthopedic self-drilling pins composed of stainless steel or titanium material.

TABLE C

| Ingredients | Amount (%) |
|---|---|
| Alexidine | 2 |
| Methanol | 11.5 |
| THF | 79 |
| Polyether Urethane | 5.5 |
| Other (e.g. excipient and/or additive) | 2 |

### Example 4

### Antimicrobial Performance of the Self-Drilling Pins Prepared as in Example 3

Description of the Test Method Used:

Uncoated control and Alexidine coated orthopedic pins of either stainless steel or titanium material were placed into screw cap tubes. Staphylococcus aureus in Trypticase Soy Broth at a concentration of 3.0×10³ CFU/ml was added to each tube at a volume large enough to cover the entire pin (7-9 ml). The pins were incubated in the inoculated broth under static conditions at 37° C. Each day, an aliquot of 100 μl was removed from the broth, serially diluted in 0.85% saline, and plated on Dey Engley Neutralizing (D/E) Agar. After 24 hours, the resulting colonies, if any, were counted and recorded. Sampling was done over a period of 11 days. On Day 11, post sampling, the pins were transferred to freshly inoculated tubes of Staphylococcus aureus containing 10³CFU/ml. Post the 24 hour incubation (Day 12), the pins were removed from the broth, gently rinsed in 0.85% saline, and placed into tubes containing D/E broth. The pins were sonicated in the neutralizing broth for 20 minutes. The sonicated broth was then sampled and plated onto D/E agar. Plates were incubated for 24 hours at 37° C. and colonies were counted and recorded.

Test Results:

Results from the stainless steel and titanium pins are shown below in Tables D and E below.

TABLE D

| | Recovered CFU/mL | | |
|---|---|---|---|
| Initial Inoculation | Uncoated | Alexidine | Alexidine |
| Concentration = | Titanium | Coated | Coated |
| 3.0 × 10³ CFU/ml | Control Pin | Titanium Pin 1 | Titanium Pin 2 |
| Day 1—initial Inoculation | | | |
| Day 2—sampling | TNTC | 0.00E+00 | 0.00E+00 |
| Day 3—sampling | 2.60E+08 | 0.00E+00 | 0.00E+00 |
| Day 4—sampling | 1.70E+08 | 0.00E+00 | 0.00E+00 |
| Days 5-7—sampling | 2.30E+08 | 0.00E+00 | 0.00E+00 |
| Day 8—sampling | 1.40E+08 | 0.00E+00 | 0.00E+00 |
| Day 10—sampling | 1.40E+08 | 0.00E+00 | 0.00E+00 |
| Day 11—sampling | TNTC | 0.00E+00 | 0.00E+00 |

Day 11—re-inoculation with 1 × 10³ CFU/ml

| | | | |
|---|---|---|---|
| Day 12—sonication to | 3.00E+06 | 0.00E+00 | 0.00E+00 |
| recover adherent biomass | | | |
| post 24 hr incubation | | | |

TABLE E

| | Recovered CFU/mL | | |
|---|---|---|---|
| | Uncoated | Alexidine | Alexidine |
| Initial Inoculation | Stainless | Coated | Coated |
| Concentration = | Steel | Stainless | Stainless |
| 3.0 × 10³ CFU/ml | Control Pin | Steel Pin 1 | Steel Pin 2 |
| Day 1—initial Inoculation | | | |
| Day 2—sampling | TNTC | 0.00E+00 | 0.00E+00 |
| Day 3—sampling | 1.20E+08 | 0.00E+00 | 0.00E+00 |
| Day 4—sampling | 1.00E+08 | 0.00E+00 | 0.00E+00 |
| Days 5-7—sampling | 6.00E+07 | 0.00E+00 | 0.00E+00 |
| Day 8—sampling | 2.00E+08 | 0.00E+00 | 0.00E+00 |
| Day 10—sampling | 8.00E+07 | 0.00E+00 | 0.00E+00 |
| Day 11—sampling | TNTC | 0.00E+00 | 0.00E+00 |

Day 11—re-inoculation with 1 × 10³ CFU/ml

| | | | |
|---|---|---|---|
| Day 12—sonication to | 3.00E+06 | 0.00E+00 | 0.00E+00 |
| recover adherent biomass | | | |
| post 24 hr incubation | | | |

### Example 5

### Minimum Inhibitory Concentration (MIC) and the Minimum Bactericidal Concentration (MBC) of Alexidine and Chlorhexidine

Description of the Test Method Used:

From the stock solutions of the drugs Alexidine and Chlorhexidine, dilution series was prepared in the wells of a 96-well plate by performing 1:1 dilutions to cover a concentration range of 0-512 ppm. Ten microliters from each of the drug concentration was mixed with 1904 of culture broth containing approximately 10⁵CFU/mL of bacteria or yeast species. The test plate was incubated for 18-24 hours after which absorbance of each well was read at 670 nm on a BioTek plate reader. The MIC value was the lowest concentration of the drug at which microbial growth was completely inhibited (with the absorbance reading at or below the reading of the drug control wells without any organisms). The wells containing growth should have had higher absorbance reading when compared to the drug control wells. After reading the absorbance for the MIC, 10 μl of each test well was plated onto the surface of D/E agar in 6 or 12 well microtiter plates to determine the MBC. The plates were incubated inverted at 37° C. for 24-48 hours after which numbers of colonies were counted. The MBC value was the lowest concentration of the drug at which no growth was observed.

Test Results:

The MIC and MBC results for Alexidine as compared to Chlorhexidine are shown in Tables F and G below. Both the MIC and MBC values for Alexidine were lower or similar to that of Chlorhexidine for most microorganisms tested indicating Alexidine is a much more potent antimicrobial agent than Chlorhexidine.

TABLE F
MIC of Alexidine versus Chlorhexidine

| | | MIC |
|---|---|---|
| | MIC Alexidine | Chlorhexidine |
| Organism | (μg/mL) | (μg/mL) |
| Staphylococcus aureus | 0.5 | 0.5 |
| Candida albicans | 1 | 2 |
| Pseudomonas aeruginosa | 8 | 8 |
| Enterococcus faecalis | 0.5 | 2 |
| Acinetobacter baumannii | 0.5 | 16 |
| Enterobacter cloacae | 2 | 2 |
| Proteus mirabilis | 1 | 8 |

TABLE G
MBC of Alexidine versus Chlorhexidine

| | MBC | MBC |
|---|---|---|
| | Alexidine | Chlorhexidine |
| Organism | (μg/mL) | (μg/mL) |
| Staphylococcus aureus | 1 | 16 |
| Candida albicans | 1 | 4 |
| Pseudomonas aeruginosa | 128 | 64 |
| Enterococcus faecalis | 2 | 64 |
| Acinetobacter baumannii | 1 | 32 |
| Enterobacter cloacae | 2 | 32 |
| Proteus mirabilis | 2 | 8 |

### Example 6

### Comparison of the Kill Time of Alexidine and Chlorhexidine

Description of the Test Method Used:

Alexidine and Chlorhexidine, both at a concentration of 128 ppm were exposed to a Gram positive bacteria (Staphylococcus aureus), a Gram negative bacteria (Pseudomonas aeruginosa), and a fungus (Candida albicans). The challenge concentration for each organism was 10⁴-10⁵ CFU/mL, and the exposure time varied from 0.5-60 minutes. Table H below shows the Time to Kill results for both Alexidine and Chlorhexidine. Complete kill of all three organisms was observed within 0.5 -1 minute of Alexidine exposure. In contrast, with Chlorhexidine it took 60 minutes before complete kill was observed for C. albicans and S. aureus, and 5 minutes for P. aeruginosa.

Test Results:

Safety Assessment

The biocompatibility and toxicity of the antimicrobial compositions of Example 3 were assessed using the six tests described below. The test results show no adverse effects and demonstrate the safety and biocompatibility of surgical devices treated with alexidine. These results surprisingly further show that the antimicrobial composition is hypoallergenic.

### Example 7

The Intracutaneous Injection Test (ISO) was performed. Test rabbits received an intracutaneous injection of the antimicrobial composition of Example 3. All test rabbits increased in body weight and showed no signs of toxicity at the 24 hour, 48 hour and 72 hour observation points.

### Example 8

The Kligman Maximization Test (ISO) was performed. The skin of guinea pigs was treated with the test article extract and exhibited no reaction to the challenge (0% sensitization).

### Example 9

A 28 day Systemic Toxicity via Intramuscular Implantation was performed. The test articles did not demonstrated any local or systemic signs of toxicity when test articles composed of the antimicrobial composition of Example 3 was implanted into the muscle tissue of five rats for 28 days.

### Example 10

The Intramuscular Implantation Test (ISO) was performed. Macroscopic evaluation of the test article implantation site indicated no significant signs of inflammation, encapsulation, hemorrhage, or necrosis. However, microscopic evaluation (histology) of these sites indicated moderate reactivity when compared to the control sites having no implantation.

### Example 11

Intravascular implantation in a Sheep Model to determine safety and efficacy was performed. The test device composed of the antimicrobial composition disclosed in Example 3 was well tolerated. All test animals remained healthy for the entire 7 and 30 day study duration and no signs of organ toxicity were observed. Alexidine-treated device was highly effective in reducing colonization by Staphylococcus aureus (the challenge organism used to infect the implantation site) on the device and the vein tissue surrounding the device. As compared to the un-treated control device, Alexidine-treated device led to 7 to 8 Log₁₀ reduction in bacterial colonization on the device and the surrounding tissue. Alexidine-treated device also led to 99% reduction in weight and 92% reduction in length of the device-associated thrombus when compared to the un-treated control device. There was also significant reduction in inflammatory response from the alexidine treated device compared to the untreated device.

### Example 12

The hemolytic index (HI) of the antimicrobial composition of Example 3 was also tested. The HI of the antimicrobial composition of Example 3 was shown to be comparable to chlorhexidine.

We claim:

1. An orthopedic device comprising an antimicrobial coating on at least one surface thereof, wherein the antimicrobial coating includes alexidine and a carrier polymer.

2. The orthopedic device of claim 1, in the form of at least one of an implant, a rod, a screw, a nail, a wire, a pin, an anchor, a plate, or a cage.

3. The orthopedic device of claim 1, wherein the antimicrobial coating further comprises a solvent selected from the group consisting of: water, alcohol, dimethyl formamide, tetrahydrofuran, ethyl acetate, butyl acetate, acetone, methyl ethyl ketone or mixtures thereof

4. The orthopedic device of claim 1, wherein the antimicrobial coating has a concentration ranging from 0.001 wt % to 4.0 wt % of alexidine.

5. The orthopedic device of claim 1, wherein the antimicrobial coating has a concentration ranging from 0.01 wt % to 2.0 wt % of alexidine.

6. The orthopedic device of claim 1, wherein the carrier polymer comprises at least one of polyurethane, polypropylene, polyester, cellulose, nylon, polysaccharide, acrylate, polyacrylonitrile, alginate, polyamide, poly (ethylene terephthalate), viscose, elastane, polyethelene oxide, ethylene methylacrylate, poly(methyl methacrylate), acrylate, cotton or mixtures thereof

7. The orthopedic device of claim 1, wherein the antimicrobial coating has a broad spectrum antimicrobial effect against the gram positive bacteria, gram negative bacteria, and fungal pathogens responsible for infections.

8. The orthopedic device of claim 1, wherein the antimicrobial coating has an antimicrobial effect on Staphylococcus species such as Staphylococcus aureus and Staphylococcus epidermidis, Candida species, Pseudomonas aeruginosa, Enterococcus species, Klebsiella species such as Klebsiella pneumoniae, Providencia stuartii, Proteus mirabilis, Enterobacter species, Acinetobacter species, and Escherichia coli.

9. The orthopedic device of claim 1, wherein the antimicrobial coating comprises alexidine at a concentration above the minimum inhibitory concentration for Staphylococcus aureus, Staphylococcus epidermidis, Candida species, Pseudomonas aeruginosa, Enterococcus species, Klebsiella pneumoniae, Providencia stuartii, Proteus mirabilis, Enterobacter species, Acinetobacter species, and Escherichia coli.

10. The orthopedic device of claim 1, wherein the antimicrobial coating prevents the formation of a biofilm on the surface of the orthopedic device.

11. The orthopedic device of claim 1, wherein the antimicrobial coating has a greater antimicrobial effect than a comparative antimicrobial coating comprising chlorhexidine as the antimicrobial agent.

12. The orthopedic device of claim 1, wherein the antimicrobial coating provides a reduced potential to cause microbial resistance than a comparative antimicrobial coating comprising chlorhexidine as an antimicrobial agent.

13. The orthopedic device of claim 1, wherein the antimicrobial coating promotes wound healing.

14. The orthopedic device of claim 1, wherein the antimicrobial coating provides a reduction in inflammatory responses including erythema, phlebitis, and/or intimal hyperplasia than a comparative antimicrobial coating comprising chlorhexidine as an antimicrobial agent.

15. The orthopedic device of claim 1, wherein the antimicrobial coating promotes bone density.

16. The orthopedic device of claim 1, wherein the antimicrobial coating has a much less potential to cause adverse reactions such as hypersensitivity and allergy than a comparative antimicrobial composition comprising chlorhexidine as the antimicrobial agent.

17. The orthopedic device of claim 1, wherein the antimicrobial coating is hypoallergenic.

18. The orthopedic device of claim 1, further comprising an adhesive material to adhere the antimicrobial coating to the orthopedic device.

19. The orthopedic device of claim 1, wherein the alexidine is in a dry state and in an amount effective to inhibit the growth of microorganisms.

20. The orthopedic device of claim 1, wherein the antimicrobial coating provides a sustained release or a controlled release of alexidine to tissues surrounding the orthopedic device.
